(19) United States
(12) Patent Application Publication
(10) Pub. No.: US 2009/0136965 A1
(43) Pub. Date: May 28, 2009

(54) Use of antibody-ligand binding to characterise diseases

(76) Inventors: Peter Lloyd, West Sussex (GB); Phil Lowe, Basel (CH); Steve Pascoe, West Sussex (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/914,152

(22) PCT Filed: May 8, 2006
(86) PCT No.: PCT/EP06/04275

§ 371 Date: Jul. 23, 2008

(30) Foreign Application Priority Data
May 10, 2005 (GB) 0509512.0

(51) Int. Cl. G01N 33/53

(52) U.S. Cl. 435/7.1

(57) ABSTRACT

We have found that when an antibody binds to (captures) its specific ligand, the antibody-ligand complex is redirected to a route of elimination which is different from that which occurs naturally for the specific ligand that is not bound to an antibody. As a consequence, the amount of antibody-bound ligand in the blood increases over time. The increase in total ligand concentration is a property that is specific to the patient to whom the antibody is administered. Accordingly, the invention provides a method for diagnosing disease in a subject and a method for identifying the most appropriate treatment for a particular patient. Patients who produce more ligand, and thus more antibody-ligand complex, may be more likely to have a disease which is predominantly driven by that ligand. These patients should respond better to a therapy targeted against that ligand. The better understanding of the underlying malfunctions in disease biology provided by the methods of the invention, in respect of the rates of production of natural ligands in health and disease, provides a logical and targeted selection of the appropriate treatments to address specific biological abnormalities.

### FIELD OF THE INVENTION

This invention relates generally to methods of using compounds or compositions for in vivo testing or in vivo diagnosis, and specifically to the use of antibody-ligand binding to characterise diseases.

### BACKGROUND OF THE INVENTION

It is known that, following administration of an antibody to a subject, the level of total target ligand is increased. See, for example, Charles P et al., J. Immunol. 163: 1521-1528 (1999).

However, there is a need in the art for further information regarding the link between increase in total ligand following administration of an antibody and the metabolic turnover of ligand. There is also a need in the art for information regarding any link between disease stratification and ligand turnover.

### SUMMARY OF THE INVENTION

We have found that when an antibody binds to (i.e., captures) its specific ligand, the antibody-ligand complex is redirected to a route of elimination which is different from that which occurs naturally for the specific ligand that is not bound to an antibody. As a consequence, the amount of antibody-bound ligand in the blood increases over time. This increase in the amount of antibody-bound ligand in the blood is not just a property of the antibody. The increase in total ligand concentration is a property that is specific to the patient to whom the antibody is administered, indicating for example the rate of production or release of the target ligand, or any changes in that production or release rate due to treatment or to other factors, such as disease, that are involved in the control of the target ligand. Individual patients will produce differing amounts of total ligand, reflecting different rates of production/release of ligand.

Accordingly, the invention provides a method for diagnosing disease in a subject. In the method of the invention, a probe dose of an antibody is administered to the subject. Then, the amount of antibody-ligand complex that is formed is measured, to determine the rate and extent of the change in this antibody-bound complex and thus measure the rate of production or release of ligand from sites in the body of the subject. The measured concentrations of antibody captured ligand, probe antibody and/or free ligand are then used to derive the rates of production and elimination of the natural ligand to help diagnose disease conditions. Taken further, the rate of production or release of ligand, as measured by this antibody induced perturbation of the system, can be used as a marker or measure of disease. For example, patients who produce more ligand, and thus more antibody-ligand complex, may be more likely to have a disease which is predominantly driven by that ligand. The disease can be any clinically meaningful measure of a disturbance from what can be considered healthy physiology and/or biochemistry. This can include specific biochemical markers though functional physiological measurements to clinical scoring systems based upon questionnaires of general health. The method of the invention is particularly useful when the circulating level of non-antibody bound target ligand cannot easily be measured by conventional means due to rapid catabolism or inactivation.

Because the influence of dose in the decline phase of total ligand vs. time can be determined, the effect of neutralising antibodies on total ligand can be detected even at high doses.

The invention also provides a method for identifying the most appropriate treatment for a particular patient. In the method of the invention, a probe dose of an antibody or cocktail of antibodies is administered to the subject. Then, the amounts of antibody-ligand complexes that are formed are measured with a suitable assay. By measuring the total level of an antibody captured ligand, one can predict the clinical outcome of a treatment. For example, patients who produce more ligand, and thus more antibody-ligand complex, may be more likely to have a disease which is predominantly driven by that ligand. These patients should respond better to a therapy targeted against that ligand. The better understanding of the underlying malfunctions in disease biology provided by the methods of the invention, in respect of the rates of production of natural ligands in health and disease, provides a logical and targeted selection of the appropriate treatments to address the specific biological abnormality.

The methods of the invention can be used in conjunction with established clinical endpoints. For example, the American College of Rheumatology (ACR) has established criteria of improvement in the treatment of rheumatoid arthritis. The methods of the invention can also be used in conjunction with laboratory procedures. For example, erythrocyte sedimentation rate (ESR) and measurements of C-reactive protein (CRP) are recognized by those of skill in the art as inflammatory markers, useful in determining inflammation during asthmatic or rheumatic responses.

In one embodiment, the administered antibody is Xolair® (omalizumab), where the level of and production rate of immunoglobulin E (IgE) correlates with the severity of asthma. The antibody omalizumab acts by capturing IgE for the treatment of asthma and allergic rhinitis.

In another embodiment, the administered antibody is ACZ885 (anti IL-1β antibody), with the production or release of IL-1β being monitored after administration and/or treatment. The antibody ACZ885 acts by capturing interleukin-1-β (IL-1β) for the treatment of respiratory diseases and rheumatoid arthritis. In the method of the invention, response to treatment is monitored against the production/release rate of IL-1β. Responders to anti IL-1β therapy are thus identified based on the production or release rate of IL-1β. In other embodiments, the administered antibodies are ABN912 (anti-monocyte chemoattractant protein), anti IL-4, anti IL-13 or anti-Thymic Stromal Lymphopoietin (anti TSLP).

### BRIEF DESCRIPTION OF THE DRAWINGS

The drawing figures depict preferred embodiments by way of example, not by way of limitations.

FIG. 1 is set of graphs showing the relationships between omalizumab, free and total IgE. Examples from three patients are given: Left panel, a placebo patient showing constant levels of IgE through the study. Total and free IgE in this case are the same as there is no omalizumab present. The centre and right panels show the effect of multiple doses (centre) and a single dose (right) of omalizumab. The upper lines are the concentrations of omalizumab; the centre lines are total IgE (free plus antibody captured complexes), the lower lines the free uncomplexed IgE.

FIG. 2 shows the relationships between ACZ885, free and total IL-1β. Until the dose of antibody is administered at time zero, total and free IL-1β are the same. Peripheral IL-1β starts at a higher concentration in the periphery as this is where it is released. When antibody is administered to the blood, it takes up to 7 days to equilibrate with the interstitial fluid. The upper line and symbol (x) represents the concentrations of antibody; the dashed line and symbol (∘) the total IL-1β (free plus antibody captured complexes), the lower lines the free uncomplexed IL-1β in the peripheral interstitial and central blood compartments.

FIG. 3 shows the relationship between exposure to total IL-1β and the improvement rate in C-reactive protein, a key component of the arthritis Disease Activity Score. The exposure to total IL-1β is measured as the area under the plasma concentration curve from the time of the first of two doses of ACZ885 to the last measured sample. The rate of improvement in the C-reactive protein (CRP) is the rate of decrease of the CRP concentration following drug administration. This is expressed as a rate constant with units of reciprocal time. CRP is a component of the rheumatoid arthritis Disease Activity Score (DAS) as given by the formula:

DAS=0.36*Logₑ(CRP+1)+0.014*GH+0.56*SQRT(T28)+0.28*SQRT(S28)+0.96

where GH is a 100 mm visual analogue general health score, T28 is the number (of 28) joints counted which are tender and S28 the number of swollen joints. The symbols in the figure are the mg/kg doses of ACZ885, all of which are better than placebo in reducing CRP.

FIG. 4 is a graph of total IL-1β in healthy (green) compared with asthmatics (blue); the asthmatics appear to have, on average, higher levels of captured ligand (on average).

FIG. 5 shows a chart following the administration of 0.3 mg/kg ABN912. The line corresponding to S3 shows an increase in total monocyte chemoattractant protein (MCP-1) that can be explained by a very rapid turnover of MCP-1. Regarding the line corresponding to S1 (free plasma MCP-1), the model of the invention predicts a transient decrease in free MCP-1 followed by a return to baseline.

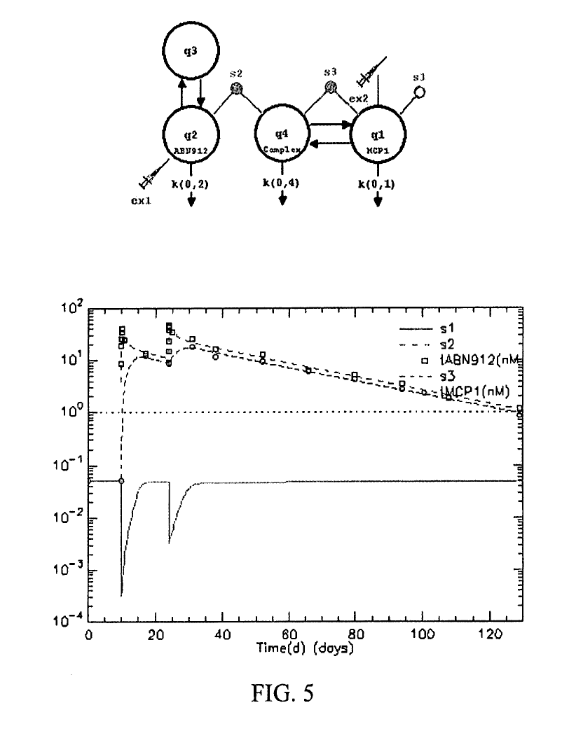

### DETAILED DESCRIPTION OF THE INVENTION

Definitions. As used herein, the term “antibody” includes, but is not limited to, polyclonal antibodies, monoclonal antibodies, humanized or chimeric antibodies and biologically functional antibody fragments sufficient for binding of the antibody fragment to the protein. See, Harlow & Lane, Antibodies: A Laboratory Manual (Cold Spring Harbor Laboratory Press, Cold Spring Harbor, N.Y., 1988). A “specific ligand” for an antibody is the composition of matter, for example in the blood of a subject, to which the antibody binds with high affinity. Many descriptions of the term specific ligand are available to those of skill in the art. See, e.g., van Oss C J, “Nature of specific ligand-receptor bonds, in particular the antigen-antibody bond.” J. Immunoassay 21(2-3):109-42 (May-August 2000).

As used herein, the term “clinical response” means any or all of the following: a quantitative measure of the response, no response, and adverse response (i.e., side effects).

Allergen exposure can cause an allergic response. During this response, T-cells (a cell type of the immune system) send a signal to B-cells (B-lymphocytes) and stimulate production of IgE antibodies—a key protein involved in the allergic cascade. Allergy Principles and Practice. 3rd Edition, Vol. 1, Elliot Middleton, ed. (Moseby Publishers, 1988); The Merck Manual of Medical Information Home Edition (Merck Research Laboratories 1997). IgE antibodies, specific to the allergen, are produced within a few weeks after exposure and released into the bloodstream. These IgE antibodies may attach to receptors on inflammatory cells such as mast cells. Unattached IgE antibodies remain free floating in the bloodstream. Taber's Cyclopedic Medical Dictionary, 16th Edition (F.A. Davis Company, 1989); Mayo Clinic Family Health Book. David E. Larson, ed. (William Morrow & Company, 1996). When an allergic individual is re-exposed to an allergen, cross-linking to IgE bound on the mast cells may occur

Xolair® is the first humanized therapeutic antibody for the treatment of asthma and the first approved therapy designed to target the antibody IgE, an underlying cause of the symptoms of allergy related asthma. See, U.S. Pat Nos. 4,816,567 and 6,329,509. The U.S. Food and Drug Administration (FDA) approved Xolair in June 2003. In addition to approval in the United States, Xolair has also received marketing license from health authorities in Australia. Xolair® binds to circulating human immunoglobulin E (IgE) at the same site as the high affinity IgE binding receptor (FcεRI), thereby preventing IgE from binding to mast cells and other effector cells. With Xolair®, fewer IgE antibodies can bind to mast cells, making IgE cross-linking less likely and inhibiting the mast cell's release of those chemicals that can cause inflammatory responses in the body.

ACZ885 (human anti-IL-1β IgG1κ antibody) is an inhibitor of IL-1β mediated eosinophilia and lung macrophage accumulation that is in Phase I development for the treatment of asthma and chronic obstructive pulmonary disease (COPD). See, published PCT patent application WO02/16436 and published U.S. patent application 2004-0063913. The use of ACZ885 also provides mechanism for treating rheumatoid arthritis. Tolchin E, Reed Life Science News (Jan. 20, 2005).

ABN912 is a fully human monoclonal antibody to Monocyte Chemoattractant Protein-1 (MCP-1) in Phase I development for the treatment of asthma and chronic obstructive pulmonary disease (COPD). See, published PCT patent application WO02/02640 and published U.S. patent application 2004-0047860.

Cuchacovich M el al., Scand. J. Rheumatol. 33: 228-232 (2004) investigated the influence of −308 tumour necrosis factor-alpha (TNF-alpha) promoter polymorphism and circulating TNF-alpha levels in the clinical response to infliximab treatment in patients with rheumatoid arthritis (RA). Infliximab is a chimeric mouse/human antibody that binds to TNF-alpha.

Several single-nucleotide polymorphisms have been identified in the human TNFα gene promoter. Among these, the −308 polymorphism generates G/G and G/A genotypes. The G/A genotype has been associated with high TNF-α production and linked to an increased susceptibility to and severity of rheumatoid arthritis (RA) in patients. Patients with the −308 TNFα gene promoter genotype G/A or with the G/G genotype were selected and received 3 mg/kg of infliximab. The authors detected a relationship between the American College of Rheumatology (ACR) criteria of improvement and increased circulating TNF-alpha levels in RA patients subjected to anti-TNFα therapy. Interestingly, while total mean TNFα levels increased with respect to basal levels in most of patients after treatment, only patients from G/A showed a statistically significant correlation between ACR50 and the increase of TNFα levels. (ACR50 is a 50% improvement in symptoms according to ACR criteria.) In the G/A genotype, mean total TNFα continues to rise throughout the study; whereas in the G/G genotype group, mean TNFα increases up to week 6 and then declines back toward baseline. The authors suggest that, taken together, these results show that a sustained increase in TNFα levels may be used to identify those patients who will present a better response to infliximab in the G/A group, i.e., in the patients that are genetically pre-disposed to a high production rate of TNFα. The authors suggest that the absence of increase in circulating TNFα levels after antibody therapy, may help to define a sub-group of RA patients with diminished response to this treatment. The inventors also suggest a significant correlation between ACR criteria improvement and increased circulating TNFα levels in patients the chimeric monoclonal antibody.

Cuchacovich M et al. used an enzyme-linked immunoassay (ELISA) to measure TNF-α levels, which allows the detection of both free and complexed TNFα. Accordingly, the authors detected increased TNFα levels that included both free and circulating TNF-α, and immune complexes of TNFα bound to the anti-TNFα monoclonal antibody. By contrast, the method of the invention comprises steps including specific assays that separate free and complexed ligand.

The following EXAMPLES are presented in order to more fully illustrate the preferred embodiments of the invention. These EXAMPLES should in no way be construed as limiting the scope of the invention, as defined by the appended claims.

### EXAMPLE I

### Omalizumab Capturing IgE in the Treatment of Allergic Rhinitis and Asthma

The binding of omalizumab to IgE can be represented chemically by the reversible reaction:

Omalizumab+IgE [symbol] Omalizumab−IgE complex

An increased amount of omalizumab drives the complexation reaction to the right, forming more drug-ligand complex (omalizumab−IgE). In doing so and in order to maintain mass balance, the concentration of the uncomplexed free IgE is reduced.

However, this simple reaction, although describing the equilibrium between the antibody (omalizumab), ligand (IgE) and the antibody captured IgE complex, does not describe the fact that all three entities have their own appearance and loss rates. Therefore a more complete model is

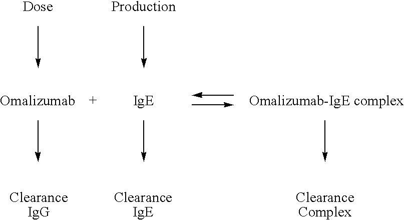

where the vertical arrows represent the input and elimination of the three entities. Accordingly, it can be seen that, given that the half-life of IgE (1-3 days) is shorter than that of IgG (23 days), the kinetic of total IgE, which is the sum of the free and the complex, is dependent upon both the rates of supply and loss of both omalizumab and IgE as well as the rates of formation and dissociation of the complex. Therefore measurement of total ligand (IgE) succinctly captures information about both drug and ligand.

The half-lives of IgG and IgE are different due to the presence in the body of a “rescue” receptor termed FcRn or neonatal receptor for the Fc portion of IgG, as discovered by Brambell (and hence also named after him). Both IgG and IgE are taken up into endothelial cells by pinocytosis. However, free IgG then binds to the Brambell receptor in the acidic conditions of the endosome, then is returned to the cell surface whereupon it is released from the Brambell receptor due to the shift back to neutral pH. Any IgG that is not bound to FcRn and IgE are degraded in the lysosomes.

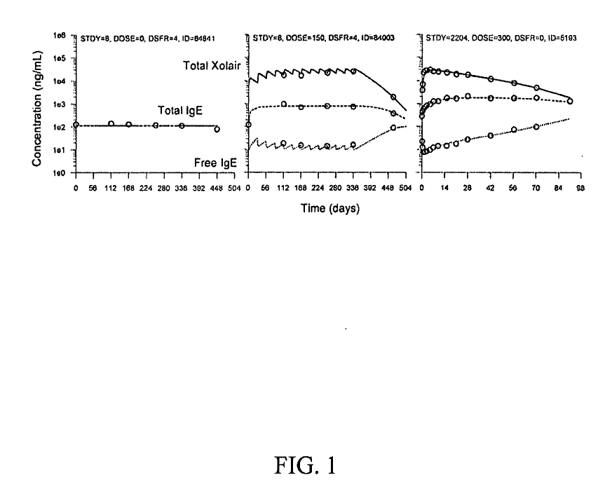

This relationship is visualised in FIG. 1. Under control (placebo) conditions the concentrations of IgE remain constant. When omalizumab is administered, either as a single or as multiple doses, free IgE is reduced in concentration whilst the total IgE increases. As can be seen from this and the equation above, whenever free ligand concentration decreases, the total antibody captured ligand increases. Conversely, when free ligand increases, total ligand decreases.

It can be seen from TABLE 1 that the concentration of free IgE is related to the clinical effectiveness of the treatment of rhinitis with omalizumab. Further, the reduction in free IgE is related to asthma exacerbations, as can be seen in TABLE 2. Therefore, since total IgE and free IgE are inversely related (as shown above), the clinical outcome is predictable based upon measurement of total IgE which, in the main, consists of antibody captured ligand. That IgE is critical in allergic rhinitis can be seen from Poole & Rosenwasser, Curr. Allergy Asthma Rep. 5(3):252-8 (May 2005), who state that “Cross-linking IgE bound to its receptor on cells by multivalent allergens initiates a chain of events resulting in allergic immune responses. Mast cells and basophils are involved in the early, immediate response, which is marked by cellular degranulation and the release of proinflammatory mediators, including histamine.”. That IgE is involved in allergic asthma can be seen from Guilbert T W et al., J. Allergy Clin. Immunol. 114(6):1282-7 (2004), who noted that total serum IgE level had the strongest correlation with aeroallergen sensitization.

TABLE 1
Nasal symptom score by free IgE concentration groups
(ITT patients with available pharmacodynamics)

| | | | | | | | |
|---|---|---|---|---|---|---|---|
| | | | | | | Estimated | |
| | | Free IgE | | | | difference | |
| | Group | concentration | | | Standard | relative to | |
| Variable | no. | group (ng/mL) | N | Mean | Deviation | group 4 | p-value |

Study 6

| | | | | | | | |
|---|---|---|---|---|---|---|---|
| Average nasal | 1 | ≦25 | 112 | 0.82 | 0.49 | −0.19 | 0.007^{a} |
| symptom severity | 2 | 25-50 | 119 | 0.86 | 0.50 | −0.14 | 0.031^{a} |
| score | 3 | 50-150 | 148 | 0.87 | 0.51 | −0.13 | 0.039^{a} |
| | 4 | >150 | 139 | 0.99 | 0.58 | | |
| Average no. of | 1 | ≦25 | 112 | 0.18 | 0.40 | −0.22 | <0.001^{a} |
| rescue | 2 | 25-50 | 120 | 0.18 | 0.34 | −0.22 | <0.001^{a} |
| antihistamine | 3 | 50-150 | 150 | 0.21 | 0.35 | −0.18 | <0.001^{a} |
| tablets per day | 4 | >150 | 141 | 0.39 | 0.60 | | |
| Proportion of days | 1 | ≦25 | 112 | 0.11 | 0.20 | −0.11 | <0.001^{a} |
| with rescue/- | 2 | 25-50 | 120 | 0.13 | 0.23 | −0.08 | 0.007^{a} |
| concomitant SAR | 3 | 50-150 | 150 | 0.15 | 0.22 | −0.06 | 0.032^{a} |
| medication use | 4 | >150 | 141 | 0.21 | 0.26 | | |

Study 7

| | | | | | | | |
|---|---|---|---|---|---|---|---|
| Average nasal | 1 | ≦25 | 113 | 0.68 | 0.44 | −0.37 | <0.001^{a} |
| symptom severity | 2 | 25-50 | 48 | 0.77 | 0.47 | −0.25 | 0.010^{a} |
| score | 3 | 50-150 | 33 | 0.86 | 0.47 | −0.20 | 0.056 |
| | 4 | >150 | 54 | 1.03 | 0.47 | | |
| Average no. of rescue | 1 | ≦25 | 113 | 0.46 | 0.80 | −1.07 | <0.001^{a} |
| antihistamine tablets | 2 | 25-50 | 49 | 0.58 | 0.70 | −0.84 | <0.001^{a} |
| per day | 3 | 50-150 | 33 | 0.87 | 1.06 | −0.64 | 0.008^{a} |
| | 4 | >150 | 54 | 1.49 | 1.59 | | |
| Proportion of days | 1 | ≦25 | 113 | 0.22 | 0.26 | −0.27 | <0.001^{a} |
| with rescue/ | 2 | 25-50 | 49 | 0.27 | 0.22 | −0.22 | <0.001^{a} |
| concomitant SAR | 3 | 50-150 | 33 | 0.37 | 0.33 | −0.13 | 0.041^{a} |
| medication use | 4 | >150 | 54 | 0.49 | 0.28 | | |

^{a}p < 0.05. p-values are from ANCOVA with the terms for dosing schedule and baseline IgE.

TABLE 2
Incidence of asthma exacerbations during steroid reduction phase by free
IgE concentration groups (ITT patients with available pharmacodynamics)

| | | | | Relative frequency | | | | | Estimated | |
|---|---|---|---|---|---|---|---|---|---|---|
| | | Free IgE | | distribution of number of asthma | | | | | odds ratio | |
| | Group | concentration | | exacerbation episodes | | | | | relative to | |
| Variable | no. | group (ng/mL) | n | 0 | 1 | 2 | 3 | 4 | group 4 | p-value |

Study 008

| | | | | | | | | | | |
|---|---|---|---|---|---|---|---|---|---|---|
| Number of asthma | 1 | ≦25 | 202 | 0.832 | 0.144 | 0.015 | 0.000 | 0.010 | 1.976 | 0.009 |
| exacerbation | 2 | 25-50 | 58 | 0.776 | 0.138 | 0.035 | 0.000 | 0.052 | 1.174 | 0.653 |
| episodes (Steroid | 3 | 50-150 | 49 | 0.816 | 0.102 | 0.041 | 0.000 | 0.041 | 1.731 | 0.188 |
| Reduction Phase) | 4 | >150 | 178 | 0.736 | 0.180 | 0.062 | 0.000 | 0.022 | | |

Study 010

| | | | | | | | | | | |
|---|---|---|---|---|---|---|---|---|---|---|
| Number of asthma | 1 | ≦25 | 181 | 0.862 | 0.077 | 0.039 | 0.006 | 0.017 | 3.085 | <0.001 |
| exacerbation | 2 | 25-50 | 42 | 0.738 | 0.167 | 0.048 | 0.000 | 0.048 | 1.314 | 0.518 |
| episodes (Steroid | 3 | 50-150 | 11 | 0.818 | 0.182 | 0.000 | 0.000 | 0.000 | 2.512 | 0.273 |
| Reduction Phase) | 4 | >150 | 82 | 0.659 | 0.244 | 0.073 | 0.012 | 0.012 | | |

Estimated odds ratio = {Prob(Y ≦ j | Free IgE group i)/[1 − Prob(Y ≦ j | Free IgE group i)]}/{Prob(Y ≦ j | Free IgE group 4)/[1 − Prob(Y ≦ j | Free IgE group 4)]} where Y is the number of asthma exacerbations episodes.

### EXAMPLE II

### ACZ885 Capturing IL-1β in the Treatment of Arthritis

The binding of ACZ885 to IL-1β can be represented chemically by the reaction:

ACZ885+IL-1β [symbol] ACZ885−IL-1β complex

Therefore, an increased amount of drug ACZ885 drives the complexation reaction to the right, forming the drug-ligand complex. In doing and in order to maintain mass balance, the concentration of the uncomplexed IL-1β is reduced.

However, this simple reaction, although describing the equilibrium between the antibody, ligand (IL-1β) and the antibody captured IL-1β complex, does not describe the fact that all three entities have their own appearance and loss rates and that there is distribution of both the antibody and the ligand between central plasma, to which the antibody is administered, and peripheral interstitial fluid into which the ligand is released. Therefore a more complete model is

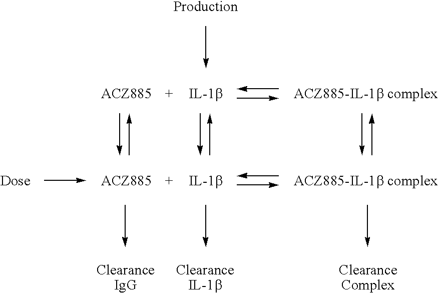

where the vertical arrows represent the input, distribution equilibration and elimination of the three entities. Accordingly, it can be seen that, since the loss rate of free IL-1β is far faster than that of IgG or the complex, the concentrations of total IL-1β (which is the sum of the free and the complex) increases dramatically, dependent upon the rates of supply and loss of both antibody (ACZ885) and ligand (IL-1β) as well as the rates of formation and dissociation of the complex.

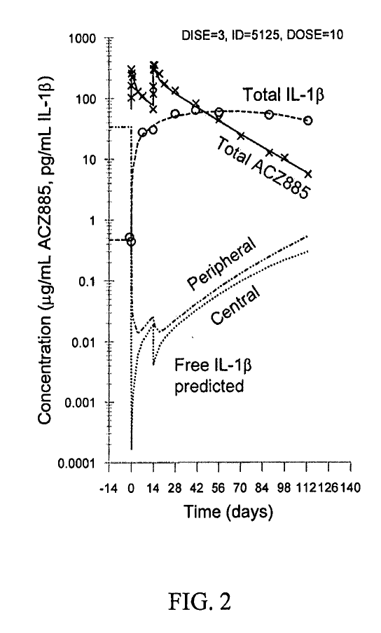

This relationships are visualised in FIG. 2. Under control (placebo) conditions the concentrations of IL-1β remain constant. When ACZ885 is administered the free IL-1β is predicted to be reduced whilst the (measured) total IL-1β increases. As can be seen from this and the equation described above, whenever the free ligand concentration is decreased, the total antibody captured ligand increases.

This EXAMPLE further illustrates the power of the invention in that here, the concentrations of the free ligand (IL-1β) could not be measured due to lack of assay sensitivity. However, from the binding relationship between the antibody and ligand, the concentrations of the free ligand can readily be inferred from the available measurements of antibody and total ligand.

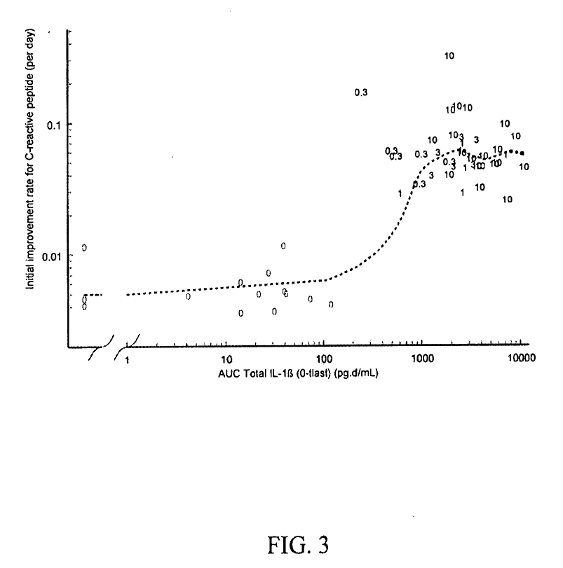

It can be seen from FIG. 3 that the measurement of total IL-1β is related to a major element of the clinical score used to quantify clinical effectiveness of the treatment of rheumatoid arthritis. Therefore, measures of exposure to cytokines such as total IL-1β which, in the main, consists of antibody captured ligand, enable the prediction of clinical responsiveness to inflammatory disorders such as asthma and rheumatoid arthritis.

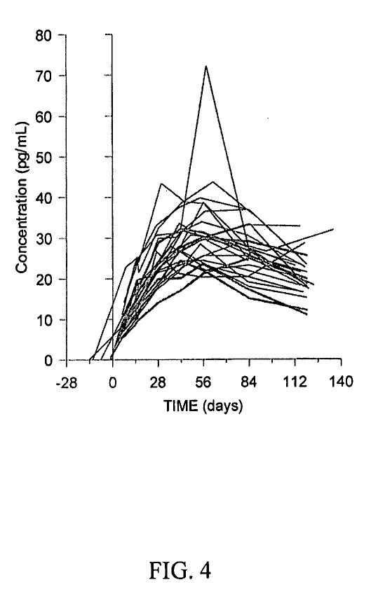

In one embodiment, a correlation for total IL-1β AUC versus ability to respond to allergen challenge is produced, using a simple area under the FEV1 curve to quantitate the effectiveness. FIG. 4 is a graph of total IL-1β in healthy (green) compared with asthmatics (blue); the asthmatics appear to have higher levels of captured ligand (on average). Accordingly, in this embodiment, the variation in the total IL-1β correlates with the effectiveness of ACZ885 in ameliorating the change in FEV1 induced by the allergen challenge.

In another embodiment, replacing erythrocyte sedimentation rate (ESR) with measurements of C-reactive protein (CRP) is performed to make the overall DAS follow this marker, which is significantly improved under ACZ885 treatment. Both CRP and ESR are inflammatory markers. CRP is sometimes used in the DAS instead of ESR as a marker of acute inflammation, so the two measurements can be reasonably substituted. In the mechanism of action of ACZ885, binding IL-1b reduces the inflammatory markers (CRP and ESR). The tender and swollen joint counts and pain scores appear not to be affected so soon, but start to reduce more slowly. In yet another embodiment, the CRP and ESR measurements are merged to determine the DAS score.

In summary, the ability to affect and/or measure the primary biomarker of ACZ885 administration (free ligand) is sensitive not only to binding affinity of the antibody, but also to the ligand concentration, turnover and expression.

### EXAMPLE III

### ABN912 Binding to MCP-1

Antibody—ligand interactions are extremely complex and dependent on the concentration and turnover of the target ligand. As a probe, ABN912, which effectively binds MCP-1, has increased the understanding of the biology of MCP-1.

The equilibrium for the binding of ABN912 to MCP-1 is shown by the equation:

K_{D}=([ABN912 free][MCP-1 free])/[ABN912−MCP-1 complex]

At equilibrium: Kₒₙ.ABN912.MCP-1=k_{off}.[complex], since

$${K}_{D} = \frac{{K}_{off}}{{K}_{on}} = \frac{ABN 912 \cdot MCP - 1 ( pM )}{[ complex ]}$$

Following administration of ABN912, and ABN912 binding to MCP-1, we found a large, rapid, dose dependent increase in total MCP-1 (mAb-MCP-1 complex), which is due to the rapid turnover of MCP-1, not an increased rate of synthesis. Plasma MCP-1 is decreased for a short time and the return to baseline levels of MCP-1 is dictated by the rapid turnover of MCP-1 (see FIG. 4). There was a decrease in serum MCP-1 to below Level of Quantification (LOQ), immediately post-dose.

The antibody ligand binding model (pharmacokinetics/pharmacodynamics (PK/PD) modelling) predicts a decrease in free MCP-1 and this prediction is confirmed in plasma.

From this EXAMPLE, it can be concluded that (1) the ability to reduce free ligand is sensitive to binding affinity of the antibody, as well as both the concentration and the turnover of the ligand; and (2) pre-clinical models (if cross-reactive) should be used to estimate: K_{D}, ligand turnover, effects on free and total ligand and homeostatic mechanisms.

Since the free target ligand MCP-1 is predicted to return to normal levels rather quickly, the method of the invention would have predicted (by 24 hrs into the treatment even with a probe dose) that the therapy was only able to neutralise the target ligand for a short period of time. Accordingly the method of the invention can discriminate between the negative therapeutic results of this EXAMPLE and the positive therapeutic results of EXAMPLE I and EXAMPLE II.

### EQUIVALENTS

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled.

We claim:

1. A method for diagnosing a disease or condition in a subject, comprising the steps of:
(a) administering an antibody to the subject;
(b) determining or calculating the total ligand concentration of the ligand to which the administered antibody binds in the subject to whom the antibody has been administered; and
(c) calculating the rate of production or release of the ligand by the subject
wherein the determination of an increase in the total ligand concentration indicates that the subject has a disease or condition which involves a change in production/release of the ligand.

2. The method of claim 1, wherein the determining step comprises determining the route of elimination of a complex between the administered antibody and the ligand of the administered antibody, wherein the route of elimination of the complex is different from that which occurs for the unbound ligand.

3. The method of claim 1, further comprising the step of:
(d) determining that the subject having a disease or condition caused by the ligand will respond to a therapy targeted against that ligand.

4. The method of claim 1, wherein the administered antibody is Xolair®.

5. The method of claim 1, wherein the administered antibody is ACZ885.

6. A method for identifying an appropriate treatment for a subject suspected of having a condition caused by a ligand to an antibody, comprising the steps of:
(a) administering an antibody or a cocktail of antibodies to the subject;
(b) determining or calculating, in the subject to whom the antibody has been administered, the total ligand concentrations of the ligands to which the administered antibodies bind,
wherein the increases in the total ligand concentrations identify the subject as one for whom reducing the concentration of the ligands is an appropriate treatment.

7. The method of claim 1, wherein the administered antibody or cocktail of antibodies comprises Xolair®.

8. The method of claim 1, wherein the administered antibody or cocktail of antibodies comprises ACZ885.
